(19) United States
(12) Patent Application Publication
(10) Pub. No.: US 2026/0232881 A1
(43) Pub. Date: Aug. 13, 2026

(54) EXTRACORPOREAL BLOOD TREATMENT DEVICE WITH SHORTENED DISINFECTION TIME

(71) Applicant: B. Braun Avitum AG, Melsungen (DE)

(72) Inventor: Waldemar Janik, Melsungen (DE)

(21) Appl. No.: 19/531,952

(22) Filed: Feb. 6, 2026

(30) Foreign Application Priority Data
Feb. 11, 2025 (DE) 10 2025 105 036.8

(51) Int. Cl. A61M 1/16; A61L 2/18; A61M 1/36

(52) U.S. Cl. CPC A61M 1/1688; A61L 2/18; A61M 1/367; A61L 2202/14; A61L 2202/17; A61M 2205/75; A61M 2209/10

(57) ABSTRACT

An extracorporeal blood treatment device includes a fluid supply unit configured to provide a temperature-controlled or temperature-controllable output fluid, preferably permeate. A treatment fluid supply unit is configured to provide a treatment fluid, preferably dialysis fluid or substitute, preferably using the output fluid. The extracorporeal blood treatment device further includes a machine front with an interface. A bypass line connects the fluid supply unit directly or indirectly to the interface, bypassing the treatment fluid supply unit. A control unit is configured to perform disinfection of the extracorporeal blood treatment device in such a way that the temperature-controlled or temperature-controllable output fluid is supplied from the fluid supply unit via the bypass line and bypassing the treatment fluid supply unit to the interface.

### CROSS-REFERENCE TO RELATED APPLICATION

This application claims priority under 35 U.S.C. § 119 to German Application No. 10 2025 105 036.8, filed on February 11, 2025, the content of which is incorporated by reference herein in its entirety.

### FIELD

The disclosure relates to an extracorporeal blood treatment device, a method for disinfecting such an extracorporeal blood treatment device, and a system comprising such an extracorporeal blood treatment device.

### BACKGROUND

Devices for extracorporeal blood treatment are generally known from the prior art. These devices are used to provide treatment fluid. The treatment fluid may comprise a dialysis fluid and a substitute. The treatment fluid can be provided by processing permeate or ultrapure water. The treatment fluid is proportioned, balanced, and filtered, for example, before being fed to an interface or connection for transfer to an extracorporeal blood line or a dialyzer. This may involve hemodiafiltration or hemodialysis, for example. After such treatment, at least the interfaces or connections for continuation, i.e., supply and return of the treatment fluid, are usually cleaned or disinfected. This disinfection process is carried out using a disinfectant fluid. In the case of disinfection with moist heat, the disinfectant fluid may be heated water/heated permeate. Alternatively or additionally, a disinfectant such as citric acid or a solution marketed under the registered trademark TIUTOL^{®} may be added. The interfaces and connections are rinsed with disinfectant fluid. This process is time-consuming and prevents the device from being used during this time.

US 5895578 A describes a method for disinfecting a dialysis machine.

EP 3765115 B1 describes a method for the local disinfection of a dialysis machine hydraulic system.

In this context, it has now become apparent that there is a need to provide an improved extracorporeal blood treatment device. In particular, there is a need to provide an extracorporeal blood treatment device with reduced disinfection or disinfection time.

### SUMMARY

It is therefore the task of the present disclosure to avoid or at least reduce the disadvantages of the prior art and, in particular, to provide an extracorporeal blood treatment device, a method for disinfecting such an extracorporeal blood treatment device, and a system with such an extracorporeal blood treatment device, with which a time-shortened disinfection of the interfaces or connections can be enabled.

Advantageous designs are explained below.

A first aspect of the present disclosure relates to an extracorporeal blood treatment device comprising: a fluid supply unit arranged to provide a temperature-controlled or temperature-controllable output fluid, preferably permeate; a treatment fluid supply unit which is designed to provide a treatment fluid, preferably dialysis fluid or substitute, preferably using the output fluid, wherein the treatment fluid supply unit preferably comprises a plurality of components, including at least one filter, in particular an ultrafiltration membrane; an interface or connection on the front of the extracorporeal blood treatment device; a bypass line that connects the fluid supply unit directly or indirectly to the interface or connection, bypassing the treatment fluid supply unit; and a control unit which is configured to perform disinfection of the extracorporeal blood treatment device in such a way that the temperature-controlled or temperature-controllable output fluid is supplied from the fluid supply unit via the bypass line and bypassing the treatment fluid supply unit to the interface or connection, so that, preferably, the temperature-controlled or temperature-controllable output fluid does not flow through the components of the treatment fluid supply unit during disinfection and thus does not flow into and through the filter, in particular the ultrafiltration membrane.

The term extracorporeal blood treatment refers in particular to methods in which blood is treated outside the body before being returned to the body. Examples of this include hemodialysis, therapeutic apheresis, hemofiltration, and hemodiafiltration.

The term “treatment fluid supply unit” refers in this context to a device that is designed to provide the treatment fluid, in particular dialysis fluid and/or substitute, or to produce it from permeate or ultrapure water. The treatment fluid supply unit may comprise one or more of the following: one (ultra) filter, preferably at least two (ultra) filters, a balancing device, a pump, a reservoir for an output fluid that is further processed into the treatment fluid, an interface for such an output fluid, valves, and lines. The treatment fluid supply unit may be distributed across several components. The treatment fluid supply unit or its elements/components can be controlled or operated via a control unit.

The interface or connection for forwarding and returning the treatment fluid refers in particular to a receptacle into which a tube can be inserted for connection to a dialyzer or for connection to an extracorporeal blood circulation system. The interface or connection can still be closed with a cap if, for example, it is not needed or the interface/connection is being disinfected. The interface or connection is located on the front of the machine, as shown in the disclosure. In this context, the machine front refers in particular to a front surface of the extracorporeal blood treatment device. The front surface may comprise any surface or any plurality of surfaces of the extracorporeal blood treatment device.

The term “fluid supply unit” refers in particular to a device that is designed to provide a temperature-controlled or temperature-controllable output fluid, preferably permeate/ultrapure water. The fluid supply unit may comprise one or more of the following: a reservoir for the output fluid, a pump, a heating element, valves, lines, etc. The fluid supply unit or its elements/components may be controlled or operated via a control unit. The output fluid may comprise permeate and, if necessary, disinfectants such as citric acid or a solution marketed under the registered trademark TIUTOL^{®}. The fluid supply unit may be distributed across several components or combined into one. In principle, the extracorporeal blood treatment device according to the disclosure may comprise a (single) fluid supply unit which can provide the temperature-controlled or temperature-controllable output fluid, in particular permeate/ultrapure water, both for the production of the treatment fluid in the treatment fluid supply unit and as a disinfectant fluid for use in thermal disinfection, if necessary in the case of the addition of a disinfectant in the context of combined thermal and chemical disinfection. Alternatively, however, it is also conceivable that two separate or independent fluid supply units are provided, of which a first fluid supply unit supplies a fluid for producing the treatment fluid and of which a second fluid supply unit supplies a (disinfectant) fluid for thermal disinfection or, if necessary, for combined thermal and chemical disinfection.

In this context, the term “bypass line” refers in particular to a line that connects the fluid supply unit to at least one interface/at least one connection on the front of the machine. The bypass line may comprise several lines. The at least one interface/the at least one connection may be connected to a further line which is directly or indirectly connected to the bypass line. The bypass line is arranged and designed to bypass the treatment fluid supply unit so that the temperature-controlled or temperature-controllable output fluid/disinfectant fluid does not have to flow through the components of the treatment fluid supply unit and does not flow through the components of the treatment fluid supply unit during disinfection. The bypass line may, for example, comprise a separate line leading directly from the fluid supply unit to the at least one interface/connection. Alternatively, the bypass line may comprise line sections that the fluid supply unit uses to connect to the at least one interface. The line sections may represent supply or discharge lines to/from the treatment fluid supply unit. The bypass line may, for example, comprise a return line/return lines connecting the at least one interface to an outlet for the treatment fluid used. In other words, the bypass line allows the treatment fluid supply unit to be bypassed so that the large-volume components of the treatment fluid supply unit, such as (ultra) filters, balancing devices, and proportioning devices, do not need to be unnecessarily filled with the temperature-controlled (output) fluid/disinfectant fluid during disinfection. In particular, the control unit may be configured to switch the single or multi-part bypass line in such a way that the at least one interface (the interfaces) or the at least one connection (the connections) are disinfected in a specific sequence and/or simultaneously. In other words, the bypass line can be divided into several paths so that different paths can be alternately flushed with the disinfectant fluid.

In this context, the term “control unit” refers in particular to a device that is designed to control or regulate components of the fluid supply unit and the treatment fluid supply unit, such as pumps, valves, heating elements, etc. The control unit can be limited to one component or distributed across several components.

The disclosure is based on the following insight: Extracorporeal blood treatment devices are usually disinfected between uses for the treatment of different patients at interfaces or connections for hygienic reasons. This disinfection process is time-consuming. Currently, essentially the entire extracorporeal blood treatment device is rinsed with temperature-controlled (disinfectant) fluid. For this purpose, in the prior art, the large-volume components of the treatment fluid supply unit, such as (ultra) filters, proportioning devices, and balancing devices, are filled with the temperature-controlled fluid and then emptied again. However, this is not absolutely necessary, as these components do not come into contact with germs or similar substances, since they are only ever exposed to fresh permeate or fresh dialysis fluid. The unnecessary disinfection process or the unnecessary disinfection of these components increases the duration of disinfection. The disclosure now proposes that, during disinfection, the treatment fluid supply unit, and in particular its large-volume components, be bypassed using a bypass line so that the temperature-controlled (disinfectant) fluid reaches the at least one interface/connection more quickly, or in other words, so that it is not necessary to fill the large-volume components of the treatment fluid supply unit. In this way, the time required for disinfection can be advantageously reduced and the availability of the extracorporeal blood treatment device can be advantageously increased. Furthermore, less water and less energy are required to heat the water, which is advantageous. This allows more patients to be treated with the same device, thereby increasing the efficiency of the device.

According to a preferred embodiment, the extracorporeal blood treatment device may further comprise a disposal interface for the disposal of the used output fluid, wherein the bypass line connects the at least one interface to the disposal interface.

In this context, the term “disposal interface” refers in particular to an interface that can be connected to a disposal container, for example. The bypass line may comprise a separate line that directs the used output fluid directly to the disposal interface. The bypass line may include drain lines used by the treatment fluid supply unit to dispose of the used treatment fluid. The latter does not adversely affect the disinfection time, as these drain lines do not flow through the large-volume components of the treatment fluid supply unit.

In this way, a complete disinfection line system can be provided in a beneficial manner.

According to a preferred embodiment, the at least one interface may comprise one or more of the following: a first output interface for forwarding the treatment fluid to a dialyzer; and/or a second output interface for forwarding the treatment fluid to an extracorporeal blood line; and/or a first input interface for returning/disposal of used treatment fluid after treatment; and/or a second input interface for returning/disposal of used treatment fluid.

The first output interface and the first input interface can be used, for example, to transport a dialysis fluid. The second output interface can be used for a substitute, for example. The second input interface can be used, for example, for the return or disposal of treatment fluid, such as priming fluid. When using the second input interface, the line system can be used to prime or vent an extracorporeal blood line system. This can be useful to prevent harmful air infusions. This means that the second input interface can be used specifically for venting or priming.

According to a preferred embodiment, the control unit may be set up to control or regulate the dwell time of the temperature-controlled fluid for performing the disinfection.

The term “dwell time” refers in particular to the period of time during which the fluid is present in or at the interface or connection. The control unit can preferably control one or more pumps and valves for directing the output fluid so that this temperature-controlled fluid/disinfectant fluid is directed to the interface/connection and remains there for a certain period of time. The fluid can flow continuously or discontinuously through the interface for disinfection. For example, the interface can be filled with the fluid once, held there for a period of time, and then pumped out again. The interface can also be continuously flushed for a certain period of time.

According to a preferred embodiment, the control unit may be set up to control and/or regulate a temperature for the (output) fluid.

The fluid supply unit preferably comprises a heating element for heating the output fluid. The control device can control and/or regulate the temperature of the output fluid via this heating element. The heating element preferably has an integrated temperature sensor for this purpose. By providing a temperature-controlled, especially heated, output fluid, disinfection can be carried out more quickly and efficiently, or thermal disinfection can be made possible in the first place.

According to a preferred embodiment, the fluid supply unit has a heating element, and the bypass line connects the heating element directly to the interface. A direct connection means that all components of the treatment fluid supply unit are bypassed and that the temperature-controlled or temperature-controllable output fluid flows directly to the interface in the line under suitable switching of two-way valves. Preferably, no complex multi-way switching valves are provided in the line between the heating element and the interface. In other words, the control unit is preferably configured in such a way that, during disinfection, the temperature-controlled or temperature-controllable output fluid, as soon as it is in the line between the heating element and the interface, flows directly to the interface without being diverted into different or multiple line sections on its way to the interface.

According to a preferred embodiment, at least one temperature sensor may be arranged in the bypass line, which is preferably used to control or regulate the temperature in the bypass line.

By placing a temperature sensor in the bypass line, the temperature can be controlled and regulated more accurately than if it were only measured at the heating element. This can advantageously increase the quality of disinfection in combination with the dwell time. Furthermore, the disinfection time can be optimized while avoiding excessively high temperatures that could damage the system. Alternatively or additionally, a temperature sensor may be arranged at or after the first input interface and/or the second input interface.

According to a preferred embodiment, the fluid supply unit may be configured to intermittently provide temperature-controlled (output) fluid.

In addition to the one-time and continuous supply mentioned above, the interface or connection can also be filled intermittently with the temperature-controlled (output) fluid, left to stand, and refilled. This can have a beneficial effect on the disinfection process.

According to a preferred embodiment, the fluid supply unit may be configured to provide a cooling fluid via the bypass line after disinfection has been performed in order to lower the temperature of at least one interface.

The cooling fluid can correspond to the output fluid at a lower temperature. Active cooling using coolant can reduce the time required for disinfecting the interface, as any necessary cooling takes place more quickly. This can increase the availability of the system. By using the bypass line, the flow through the large-volume components of the treatment fluid supply unit can be bypassed. This can have a synergistic beneficial effect on the efficiency of disinfecting the at least one interface.

According to a preferred embodiment, the extracorporeal blood treatment device may comprise at least one supply line for connecting the treatment fluid supply unit to the at least one interface.

The supply line can advantageously enable the basic function of the extracorporeal blood treatment device, namely the provision of the treatment fluid.

According to a preferred embodiment, the bypass line may differ from the supply line.

In other words, the bypass line is preferably a separate line from the supply line. This can have a beneficial effect on efficiency. For example, the length of the bypass line can be optimized so that as little volume as possible has to flow through for disinfection.

Preferably, the bypass line can be insulated from the environment so that heat losses are reduced. This can have a beneficial effect on the efficiency of the device.

According to a preferred embodiment, the treatment fluid supply unit may comprise one or more of the following: a proportioning device, an inlet of a balancing device, at least one (ultra) filter; and/or the bypass line may comprise a flushing line between the second output interface and the second input interface; and/or the bypass line may comprise a flushing line between the first output interface and the first input interface.

The term “proportioning device” refers here to a device that is designed to add a basic and an acidic component to an output fluid, such as permeate, in a controlled manner, thereby producing a treatment fluid.

The flushing lines make it possible to implement a shared disinfection circuit for multiple interfaces, which is advantageous. This can increase the efficiency of disinfection.

According to a preferred embodiment, the fluid supply unit may be configured to provide, after disinfection has been performed, a cooling fluid to lower a temperature of the at least one interface via the at least one filter and the at least one supply line.

This can have a beneficial effect on the efficiency of disinfecting the at least one interface.

According to a preferred embodiment, the fluid supply unit may comprise a container for permeate with a heating element and at least one pump.

Another aspect of the present disclosure relates to a method for disinfecting an extracorporeal blood treatment device, in particular a device described in more detail above; comprising providing a temperature-controlled or temperature-controllable output fluid; and during disinfection, feeding the temperature-controlled or temperature-controllable output fluid from a fluid supply unit of the extracorporeal blood treatment device via a bypass line of the extracorporeal blood treatment device and bypassing a treatment fluid supply unit of the extracorporeal blood treatment device to at least one interface or connection on a machine front of the extracorporeal blood treatment device; and preferably discharging the output fluid from the at least one interface.

According to a preferred embodiment, the method may further comprise the following steps: Controlling or regulating a dwell time of the output fluid; and/or controlling or regulating a temperature of the output fluid; and/or blocking a supply line of the extracorporeal blood treatment device.

The method disclosed is performed when no patient is connected to the extracorporeal blood treatment device. Accordingly, the method disclosed is not a method for surgical or therapeutic treatment of the human or animal body, nor is it a diagnostic method performed on the human or animal body.

Another aspect of the present disclosure relates to a system comprising an extracorporeal blood treatment device described in more detail above, as well as a dialyzer and/or an extracorporeal blood line or an extracorporeal tubing system.

The units according to one or more exemplary embodiments may be implemented using hardware, software, and/or a combination thereof. The units can be single-piece or multi-piece. Hardware units can be, for example, processing circuits such as a processor, a central processing unit (CPU), a controller, an arithmetic logic unit (ALU), a digital signal processor, a microcomputer, a field-programmable gate array (FPGA), a system-on-chip (SoC), a programmable logic device, a microprocessor, or any other device capable of responding to commands and executing them in a predetermined manner.

The units may comprise one or more interface circuits. In some examples, the interface circuits may include wired or wireless interfaces connected to a local area network (LAN), the Internet, a wide area network (WAN), or combinations thereof. The functionality of a specific unit of the present disclosure may be distributed across multiple units connected via interface circuits.

The units according to one or more exemplary embodiments may also include one or more storage devices. The one or more storage devices may be physical or non-transitory computer-readable storage media, such as random access memory (RAM), read-only memory (ROM), a permanent mass storage device (e.g., a hard disk drive), a solid-state device (e.g., NAND flash), and/or any other data storage mechanism capable of storing and recording data. The one storage device or the multiple storage devices may be configured to store computer programs, program code, instructions, or a combination thereof.

The explanations and advantages of individual embodiments described here also apply mutatis mutandis to the other embodiments. Various exemplary features of the embodiments may be combined as disclosed wherever technically useful and feasible.

### BRIEF DESCRIPTION OF THE DRAWINGS

The present disclosure will be explained below with the aid of figures. The following is shown in the figures below:

FIG. 1 a first schematic view of an extracorporeal blood treatment device according to the disclosure during extracorporeal blood treatment;

FIG. 2 a second schematic view of an extracorporeal blood treatment device according to the disclosure during disinfection;

FIG. 3 another schematic view of an extracorporeal blood treatment device according to the disclosure during disinfection;

FIG. 4 a diagram of the sequence of steps for disinfecting such a device; and

FIG. 5 the relationship between time and temperature during disinfection.

### DETAILED DESCRIPTION

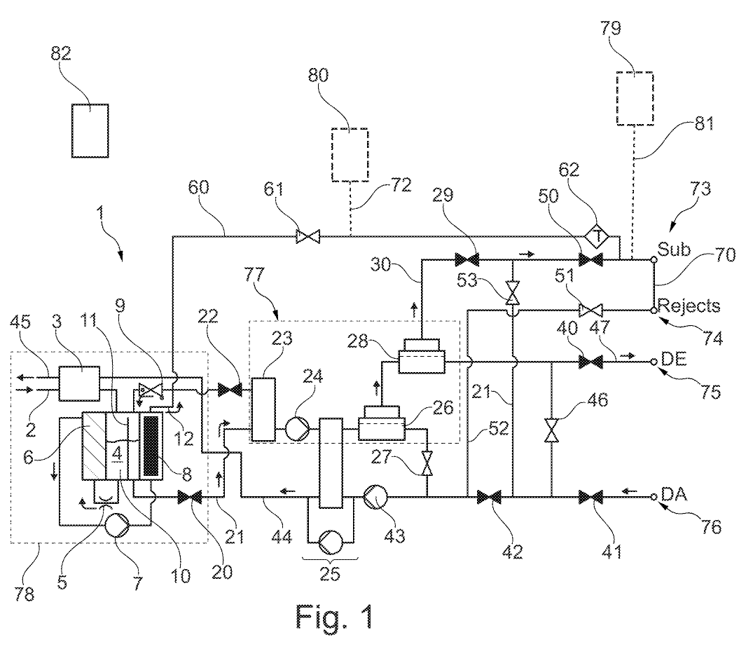

FIG. 1 shows an extracorporeal blood treatment device 1.

In the figures, white filled valves symbolize closed valves. Black filled valves, on the other hand, indicate open valves. The arrows next to the lines indicate the direction of flow.

The extracorporeal blood treatment device 1 comprises a treatment fluid supply unit 77 for supplying a treatment fluid. The treatment fluid supply unit 77 comprises a proportioning device 23, an inlet to a balancing device 25, an (ultra)filter 26, and an (ultra)filter 28.

The extracorporeal blood treatment device 1 comprises a fluid supply unit 78.

The fluid supply unit 78 comprises a water inlet 2 for supplying permeate, i.e., high-purity water. The permeate is fed into a pre-flow container 4. A degassing pump 7 conveys the permeate from the pre-flow container 4 through a throttle 5 and a degassing chamber 6 to another chamber containing a heating element 8, which heats the permeate to a desired target temperature. The negative pressure created by the throttle 5 causes the permeate to degas. The heated and degassed permeate passes through line 12 and throttle 9 into chamber 10, which is connected to the main line 21 and from which the permeate can be withdrawn for the production of dialysis fluid. If the level in chamber 10 rises, the permeate can flow back into chamber 4 via the partition wall 11. The throttle 9 ensures that the pressure required to fill the corresponding cartridge when using sodium bicarbonate (basic component) in powder form is built up.

The heated and degassed permeate flows through valve 22 into the proportioning device 23 of the treatment fluid supply unit 77. There, an alkaline and an acidic component are added to the permeate, which mainly passes through line 21, in a controlled manner, ultimately producing the finished dialysis fluid. Driven by pump 24, the dialysis fluid flows through the balancing device 25 and is filtered for the first time in the (ultra) filter 26. Valve 27 can be opened to flush filter 26.

When valve 40 is open, the dialysis fluid flows through line 47 via the dialysis fluid input DE, in this case the first output interface 75, into a dialyzer (not shown) and then leaves the dialyzer again as dialysate in order to flow via the dialysate output DA, in this case the first input interface 76, and via the open valves 41, 42, by means of pump 43 through line 44 to heat exchanger 3 and be discarded via drain 45. Line 47 may be located partly outside a housing of the extracorporeal blood treatment device 1. In particular, line 47 may continue outside the housing from DE in a dialysis fluid line (not shown) connected to the dialyzer. Line 44 may be partially located outside the housing of the extracorporeal blood treatment device 1. In particular, a dialysate line connected to the dialyzer and to DA outside the housing may be part of line 44. It should be noted that lines outside the housing, such as a dialysis fluid line or a dialysate line, should preferably be insulated to prevent heat loss. It should also be noted that, in the case of such lines provided outside the housing, a flushing bridge or a flushing line with corresponding connections to the housing may be provided. In this case, before flushing or disinfecting, the ends of the dialysate fluid line and the dialysate line connected to the dialyzer can be connected to the connections of the flushing bridge or flushing line, so that a direct flush or disinfect connection between DE and DA can be established via the dialysate fluid line, the flushing bridge or flushing line, and the dialysate line.

To perform hemodiafiltration, part of the dialysis fluid is removed. For this purpose, this fluid is conveyed through a second (ultra) filter 28 by means of a substitution pump (not shown). The substitute flows through line 30 and the open valves 29, 50 to the substitution connection Sub, in this case the second output interface 73, and can be fed from there via a dilution line outside the housing of device 1 to an extracorporeal blood line and administered to a patient by pre- or post-dilution.

When filling or flushing and/or emptying the extracorporeal tube system, it can be connected to the connection for rejects, in this case the second input interface 74. In this case, valve 51 is open so that fluid can be discharged via line 52. During hemodiafiltration, valve 51 is closed.

The sub and reject connections are connected to each other via a flushing channel 70, particularly for cleaning and disinfection purposes.

The extracorporeal blood treatment device 1 may optionally have a separate fluid supply unit 80. This supply unit 80 may comprise a pump and a container with a heating element. It may be provided that the fluid supply unit 78 only supplies fluid for producing the treatment fluid, and that a separate fluid supply unit 80 is provided which supplies fluid for disinfection/disinfectant fluid. However, it is preferable if no separate fluid supply unit 80 is provided. In other words, it is preferable if the fluid supply unit 78 supplies fluid both for producing the treatment fluid and for disinfection.

The extracorporeal blood treatment device 1 further comprises a bypass line 72, 60, which preferably connects the second output interface 73 to the fluid supply unit 78, alternatively to the fluid supply unit 80, for performing disinfection. The bypass line 72, 60 bypasses the treatment fluid supply unit 77 so that it is not flushed or disinfected with the output fluid during disinfection when the second output interface 73 is disinfected. The extracorporeal blood treatment device 1 may further optionally comprise a disposal interface 79 connected to the second output interface 73 via a bypass line 81. The extracorporeal blood treatment device 1 further comprises a control unit 82 that is configured to control the supply of the treatment fluid and the supply of the output fluid. The control unit 82 controls the dwell time of the output fluid for performing disinfection and the temperature of the output fluid. For this purpose, a temperature sensor 62 is arranged in the bypass line 60. The fluid supply unit 78 is designed to provide the disinfectant fluid once, continuously, or intermittently. The fluid supply unit 78 is further configured to provide, after disinfection has been performed, a cooling fluid for lowering a temperature of the second output interface 73 via the bypass line 72, 60. In the event that bypass lines 72 and 81 are present, these preferably each comprise valves (not shown).

In FIG. 1, the valves are connected in such a way that the device only supplies the treatment fluids in the form of dialysis fluid and substitute. The valve circuit thus corresponds to the circuit during extracorporeal blood treatment.

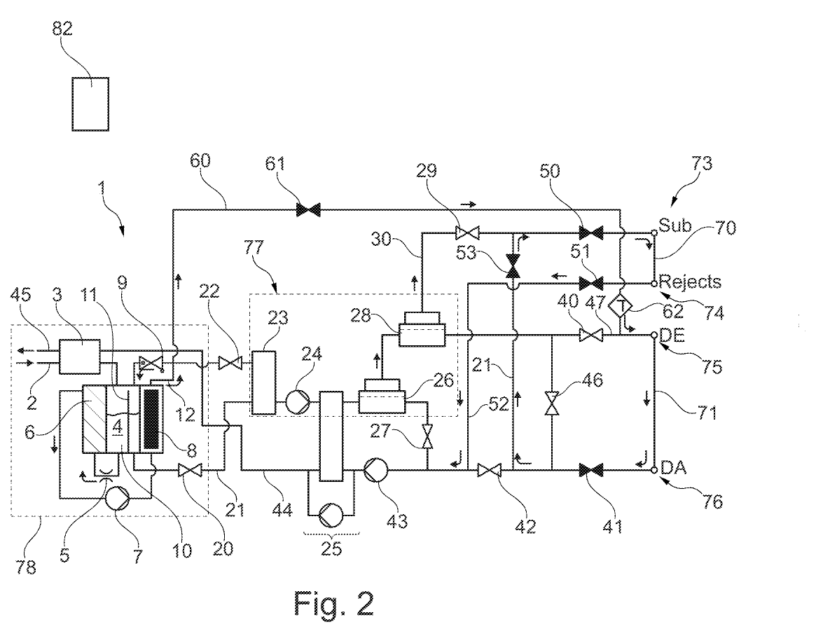

FIG. 2 shows another schematic view of the extracorporeal blood treatment device 1 according to the disclosure. The extracorporeal blood treatment device 1 is currently in disinfection mode. The heating element 8 heats the permeate. Valves 20 and 22 are closed and pump 24 is inactive. Due to the excess pressure in line 12 and the open valve 61, the heated water can flow through line 60 directly to the dialysis fluid connection DE, in this case the first output interface 75. Line 60 is preferably insulated to prevent heat loss. The sensor 62 can be used to measure the temperature and, if necessary, adjust it using the heating element 8. The heated fluid flows through the flushing line 71 and line 21 to the substitute connection, here the second output interface 73, and via the flushing channel 70 to the rejects, here the second input interface 74, before being forwarded to the drain 45. In this way, the dialyzer connections DE 75 and DA 76 as well as the connections for the extracorporeal blood tube system Sub 73 and rejects 74 are flushed with disinfectant fluid (in this case, heated permeate). Since the rest of the hydraulic system, which has a high volume due to, for example, the proportioning device 23 and the balancing device 25 as well as the two filters 26, 28, is bypassed, a shortened hot disinfection can be carried out. Alternatively, the temperature sensor 62 may be located in the line 52, or an additional temperature sensor may be provided there, so that the temperature can be measured or estimated throughout the entire critical range. The flow of disinfectant fluid through line 60 may be intermittent. If the temperature drops to the critical range, valve 61 can be opened so that hotter disinfectant fluid can flow in. The disinfectant fluid can reach the critical area due to excess pressure in line 12. Alternatively, pump 43 can suck in the disinfectant fluid. Alternatively, the pump of balancing unit 25 can suck in the disinfectant fluid. Alternatively, line 60 can branch off after temperature sensor 62, for example. The first part can then flow into the substitute line 30 and the second part into the dialysis fluid line 47. Through clever valve switching, the second output interface 73 and the second input interface 74, as well as the first output interface 75 and the first input interface 76, can be alternately flushed with the disinfectant fluid, thereby preventing heat loss.

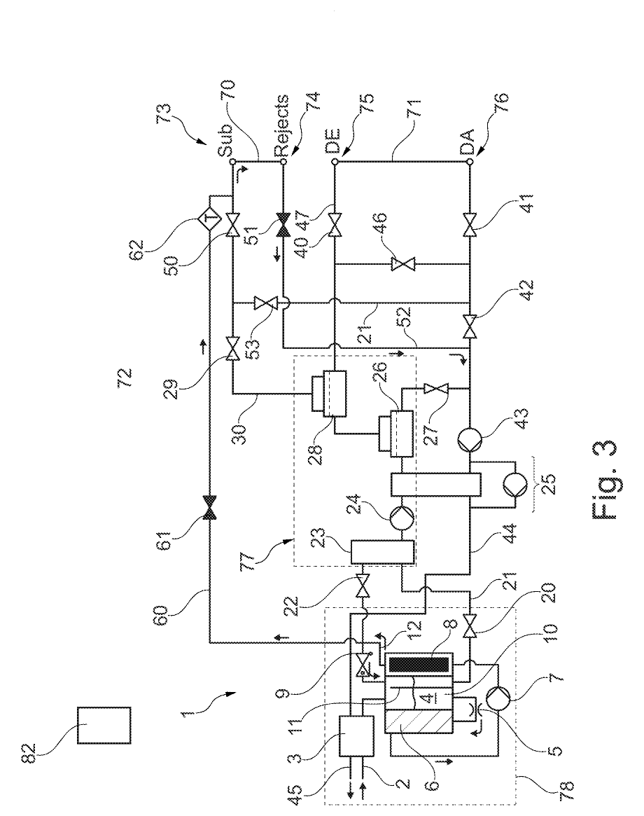

FIG. 3 shows another view of a device 1 according to the disclosure for extracorporeal blood treatment. Here, bypass line 60 flows directly into substitute line 30. When valve 50 is closed, the disinfectant fluid flows through the second output interface 73 and reaches the second input interface 74 via the flushing line 70. In this arrangement, the first output interface 75 and the first input interface 76 are not disinfected, as they may not need to be disinfected if the treatment fluid always flows from the first output interface 75 (through the dialyzer) to the first input interface 76 during treatment.

FIGS. 1 through 3 show examples of hemodiafiltration (HDF) circuits. Alternatively, the device could also be used for hemodialysis hydraulics (HD). In contrast to HDF, the following components would essentially be omitted in classic HD hydraulics: 28, 3029, 50, 73, 70, 74, 51, 53, 21, 52. As shown in FIG. 2, in this case, the bypass line 60 would flow directly into line 47, and the disinfectant fluid would flow through 47, 75, 71, 76, and 45.

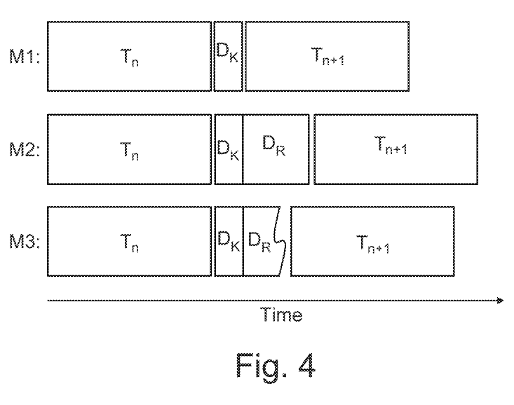

FIG. 4 is a diagram showing the sequence of steps for disinfecting such a device. In mode M1 after a therapy Tn, a brief hot disinfection of the critical area DK is performed before the next therapy Tn + 1 is carried out. In M2, brief hot disinfection of the critical area DK is the first sequence of a complete disinfection process, which is followed by disinfection of the remaining hydraulics DR. M3 shows how DR is discontinued in order to start a new therapy Tn + 1 prematurely.

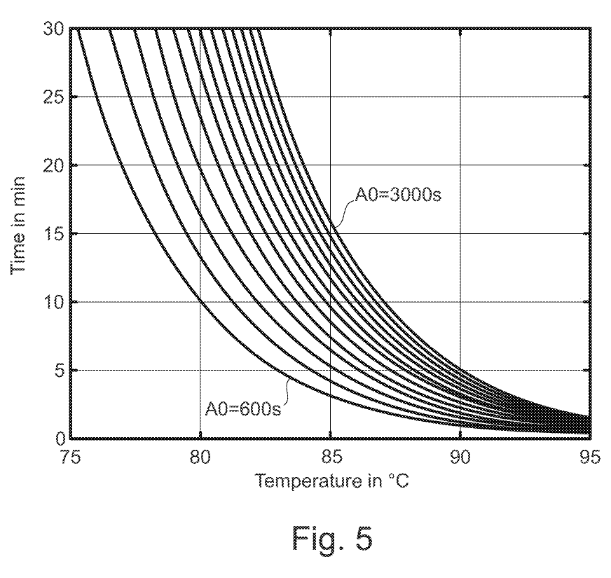

FIG. 5 illustrates the relationship between time and temperature during disinfection. Two parameters can be set for disinfection with moist heat. These are the disinfection temperature and the disinfection duration. In addition, the temperature sensitivity of the microorganism in question plays a role. The relationship between these parameters is described in the A0 concept. The A0 method evaluates the process of thermal disinfection according to its effectiveness. Disinfection with an A0 value of 600 s is specified as the minimum value for surgical instruments. This value should also be achievable during dialysis when disinfecting the hydraulic system of the device described above. Here, the exposure time is shown for different A0 values between 600 s and 3000 s (intervals of 200 s) as a function of temperature. For example, to achieve an A0 value of 600 s at a temperature of 90°C, an exposure time of only 1 min is required. An A0 value of 3000 s is reached after 5 min. For example, the second output interface 73 is the most critical, as it creates a direct connection to the blood via pre- or post-dilution. For security reasons, the second output interface 73 can be disinfected with a higher target value for A0 than, for example, the first input interface 76. With the appropriate choice of flow sequence, a temperature loss at, for example, the first input interface 76 can be tolerated, as a lower A0 value may be required there than at the second output interface 73.

### List of reference signs

1 Extracorporeal blood treatment device

2 Permeate inlet

3 heat exchanger

4 Pre-flow container

5 Throttle

6 Degassing chamber

7 Degassing pump

8 Heating element

9 Throttle

10 Removal chamber

11 Partition wall

12 Line

20 Main flow valve

21 Dialysate-carrying or permeate-carrying line

22 Dialysate-carrying line

23 Proportioning device

24 Pump

25 Balancing device

26 Filter

27 Valve

28 Filter

29 Valve

30 Substitute line

40 Dialysis fluid input valve

41 Dialysate output valve

42 Valve

43 Pump

44 Dialysate Line

45 Drain

46 Bypass valve

47 Dialysis fluid line

50 Substitute valve

51 Valve for rejects

52 Line for rejects

53 Bypass valve substitute

60 Line for hot fluid

61 Valve

62 Temperature sensor

70 Substitute flushing line

71 Dialysate flushing line

72 Bypass line

73 Second output interface

74 Second input interface

75 First output interface

76 First input interface

77 Treatment fluid supply unit

78, 80 Fluid delivery unit

79 Disposal interface

81 Bypass line

82 Control unit

1. An extracorporeal blood treatment device comprising:
a fluid supply unit configured to provide an output fluid that is temperature-controlled or temperature-controllable;
a treatment fluid supply unit configured to provide a treatment fluid;
a front panel with at least one interface on the front panel;
a bypass line that connects the fluid supply unit to the at least one interface directly or indirectly, bypassing the treatment fluid supply unit; and
a control unit,
the control unit configured to perform a disinfection of the extracorporeal blood treatment device in such a way that the output fluid is supplied from the fluid supply unit via the bypass line and bypassing the treatment fluid supply unit to the at least one interface.

2. The extracorporeal blood treatment device according to claim 1, further comprising a disposal interface for disposal of output fluid after the output fluid is used, wherein the at least one interface is connected to the disposal interface.

3. The extracorporeal blood treatment device according to claim 1,
wherein the at least one interface comprises at least one of:
a first output interface for forwarding the treatment fluid to a dialyzer;
a second output interface for forwarding the treatment fluid to an extracorporeal blood line;
a first input interface for returning a used treatment fluid; or
a second input interface for returning the used treatment fluid.

4. The extracorporeal blood treatment device according to claim 3, wherein:
the bypass line comprises a flushing line between the second output interface and the second input interface; and/or
the bypass line comprises a flushing line between the first output interface and the first input interface.

5. The extracorporeal blood treatment device according to claim 1, wherein the control unit is configured to control or regulate a dwell time of the output fluid for performing the disinfection.

6. The extracorporeal blood treatment device according to claim 1, wherein the control unit is configured to control or regulate a temperature of the output fluid.

7. The extracorporeal blood treatment device according to claim 6, wherein at least one temperature sensor is arranged for controlling or regulating temperature in the bypass line.

8. The extracorporeal blood treatment device according to claim 1, wherein the fluid supply unit is configured to intermittently provide the output fluid.

9. The extracorporeal blood treatment device according to claim 1, wherein the fluid supply unit is configured, after performing the disinfection, to provide a cooling fluid via the bypass line to lower a temperature of the at least one interface.

10. The extracorporeal blood treatment device according to claim 1, further comprising at least one supply line for connecting the treatment fluid supply unit to the at least one interface.

11. The extracorporeal blood treatment device according to claim 10, wherein the bypass line differs from the at least one supply line.

12. The extracorporeal blood treatment device according to claim 1, wherein the treatment fluid supply unit comprises at least one of:
a proportioning device,
an inlet of a balancing device, or
at least one filter.

13. The extracorporeal blood treatment device according to claim 12, further comprising at least one supply line for connecting the treatment fluid supply unit to the at least one interface, wherein the treatment fluid supply unit comprises at least one filter, and the fluid supply unit is configured, after performing the disinfection, to provide a cooling fluid to lower a temperature of the at least one interface via the at least one filter and the at least one supply line.

14. The extracorporeal blood treatment device according to claim 1, wherein the fluid supply unit comprises a container for permeate with a heating element and at least one pump.

15. A system comprising:
the extracorporeal blood treatment device according to claim 1; and
at least one of:
a dialyzer, or
an extracorporeal blood line.

16. The extracorporeal blood treatment device according to claim 1, wherein the output fluid is permeate.

17. The extracorporeal blood treatment device according to claim 1, wherein the treatment fluid is dialysis fluid or a substitute.

18. A method for disinfecting an extracorporeal blood treatment device comprising the steps of:
providing an output fluid that is temperature-controlled or temperature-controllable;
supplying the output fluid from a fluid supply unit via at least one bypass line; and
bypassing a treatment fluid supply unit to at least one interface on a machine front of the extracorporeal blood treatment device.

19. The method according to claim 18, further comprising the step of discharging the output fluid from the at least one interface.

20. The method according to claim 18, further comprising at least one of the steps of:
controlling or regulating a retention time of the output fluid;
controlling or regulating a temperature of the output fluid; or
blocking a supply line.
